SEQUENCE LISTING (1 sequence)

SEQ ID NO: 1 - DNA, 4868 bp, Homo sapiens
ctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaacaaagcaggctttaaaggaaccaattcagtcgacggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggaagcttggatccccaccatggaggccgtggagaccggggaacggcccaccttcggagcctgggactacggggtctttgccctcatgctcctggtgtccactggcatcgggctgtgggtcgggctggctcggggcgggcagcgcagcgctgaggacttcttcaccgggggccggcgcctggcggccctgcccgtgggcctgtcgctgtctgccagcttcatgtcggccgtgcaggtgctgggcgtgccgtcggaggcctatcgctatggcctcaagttcctctggatgtgcctgggccagcttctgaactcggtcctcaccgccctgctcttcatgcccgtcttctaccgcctgggcctcaccagcacctacgagtacctggagatgcgcttcagccgcgcagtgcggctctgcgggactttgcagtacattgtagccacgatgctgtacaccggcatcgtaatctacgcaccggccctcatcctgaaccaagtgaccgggctggacatctgggcgtcgctcctgtccaccggaattatctgcaccttctacacggctgtgggcggcatgaaggctgtggtctggactgatgtgttccaggtcgtggtgatgctaagtggcttctgggttgtcctggcacgcggtgtcatgcttgtgggcgggccccgccaggtactcacgctggcccagaaccactcccggatcaacctcatggactttaaccctgacccgaggagccgctatacattctggacttttgtggtgggtggcacgttggtgtggctctccatgtatggcgtgaaccaggcgcaggtgcagcgctacgtggcttgccgcacagagaagcaggccaagctggccctgctcatcaaccaggtcggcctgttcctgatcgtgtccagcgctgcctgctgtggcatcgtcatgtttgtgttctacactgactgcgaccctctcctcctggggcgcatctctgccccagaccagtacatgcctctgctggtgctggacatcttcgaagatctgcctggagtccccgggcttttcctggcctgtgcttacagtggcaccctcagcacagcatccaccagcatcaatgctatggctgcagtcactgtagaagacctcatcaaacctcggctgcggagcctggcacccaggaaactcgtgattatctccaaggggctctcactcatctacggatcggcctgtctcaccgtggcagccctgtcctcactgctcggaggaggtgtccttcagggctccttcaccgtcatgggagtcatcagcggccccctgctgggagccttcatcttgggaatgttcctgccggcctgcaacacaccgggcgtcctcgcgggactaggcgcgggcttggcgctgtcgctgtgggtggccttgggcgccacgctgtacccacccagcgagcagaccatgagggtcctgccatcgtcggctgcccgctgcgtggctctctcagtcaacgcctctggcctcctggacccggctctcctccctgctaacgactccagcagggcccccagctcaggaatggacgccagccgacccgccttagctgacagcttctatgccatctcctatctctattacggtgccctgggcacgctgaccactgtgctgtgcggagccctcatcagctgcctgacaggccccaccaagcgcagcaccctggccccgggattgttgtggtgggacctcgcacggcagacagcatcagtggcccccaaggaagaagtggccatcctggatgacaacttggtcaagggtcctgaagaactccccactggaaacaagaagccccctggcttcctgcccaccaatgaggatcgtctgtttttcttggggcagaaggagctggagggggctggctcttggaccccctgtgttggacatgatggtggtcgagaccagcaggagacaaacctctgagaattctgcagatatccagcacagtggcggccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgtccctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagacccggtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

(19) United States
(12) Patent Application Publication
(10) Pub. No.: US 2021/0244832 A1
(43) Pub. Date: Aug. 12, 2021

(54) BIO-IMAGING PROBES AND METHODS FOR NON-INVASIVE DETECTION OF HUMAN UTERINE SARCOMAS

(71) Applicant: THE BOARD OF TRUSTEES FO THE UNIVERSITY OF ILLINOIS, Urbana, IL (US)

(72) Inventors: Ayman Al-Hendy, Chicago, IL (US); Natalia Garcia, Chicago, IL (US)

(21) Appl. No.: 17/111,485

(22) Filed: Dec. 3, 2020

Related U.S. Application Data
(63) Continuation-in-part of application No. 17/105,966, filed on Nov. 27, 2020.
(60) Provisional application No. 62/946,958, filed on Dec. 11, 2019.
(60) Provisional application No. 62/941,676, filed on Nov. 27, 2019.

(51) Int. Cl. A61K 51/12; A61B 6/03; A61B 6/00; A61K 9/00; A61P 35/00; C12N 15/86; C07K 14/705; C07K 14/005

(52) U.S. Cl. CPC A61K 51/1203; A61B 6/037; A61B 6/50; A61K 9/0019; A61P 35/00; C12N 15/86; C12N 2710/10031; C07K 14/005; A61B 6/508; C12N 2710/10043; C12N 2710/10022; C12N 2830/008; C07K 14/705

(57) ABSTRACT

This invention provides compositions of a viral vector system containing a survivin promotor configured for expressing a reporter gene, wherein the reporter gene is differentially expressed in benign and malignant uterine masses and malignant uterine sarcomas can be bioimaged by preferential labeling with a radiotracer.

### RELATED APPLICATIONS

This application claims priority to U.S. Provisional Patent Application No. 62/941,676 filed Nov. 27, 2019, U.S. Provisional Patent Application No. 62/946,958 filed on Dec. 11, 2019, and U.S. patent application Ser. No. 17/105,966 filed on Nov. 27, 2020. All are hereby incorporated in their entireties.

### TECHNICAL FIELD

The disclosure relates to reagents and methods for screening or identifying for differential diagnosis of malignant uterine masses.

### BACKGROUND

Uterine sarcomas are a highly aggressive gynecologic malignancies. Annual incidence in the United States alone is 0.8 per 100,000 women. Nineteen million women present with uterine masses annually. Such masses can be benign fibroids or malignant sarcomas, and roughly eighty percent of uterine masses detected are suspicious. There are, however, no known tools for distinguishing between benign fibroid vs. malignant sarcoma without surgical intervention in humans. Further, delayed diagnosis of patients usually leads to poor prognosis.

Uterine leiomyosarcoma (LMS) is the most common type of uterine sarcoma, accounting for approximately 42-60% of all uterine sarcomas. Uterine fibroids (LM) are the most common benign pelvic tumors in women, this pathology can be present in up to 80% of women by the age of 50. Both tumors present the same clinical symptoms (abnormal uterine bleeding, pelvic mass and pelvic pain) LMS has been challenging to diagnose before surgery due to limitations in clinical and radiographic predictors as well as lack of reliable serum or urinary biomarkers. The majority of women with uterine masses do indeed have benign fibroids. However, currently, it is a major challenge in gynecologic surgery to differentiate between LM vs. LMS before surgery.

Patients are typically treated by open radical surgery & adjuvant therapy, because a uterine mass cannot be pre-surgically ascertained as benign or malignant with currently available technologies and the negative prospect for patients with malignancy. Fibroids are the most common benign growth in women of reproductive age. Uterine masses can be found in almost 70-80% in women by age of 50 Y. Patients cannot currently be treated medically without minimally invasive surgery, since non-surgical diagnostic capabilities for non-invasive differentiation between human uterine fibroids (benign) versus sarcoma (malignant) in women do not currently exist. Thus, practitioners must perform invasive surgery to treat patients as the current standard. Further, there are no known biomarkers that can distinguish between uterine sarcoma versus fibroid, nor is there a simple, effective screening method to differentiate benign and malignant uterine sarcomas in humans.

Differential diagnosis of uterine masses, a very common diagnosis in reproductive age women, includes uterine fibroids (benign) versus leiomyosarcoma (highly malignant). Current imaging techniques cannot distinguish between these two disease entities, and there is no available biomarkers or biopsy-based methodology. Most of these patients currently are offered open surgery (laparotomy) where the pathological nature of the uterine mass can be firmly ascertained (via frozen section pathological assessment) and treatment is also rendered

Prior studies have involved using Adenovirus Survivin Luciferase (Ad-Sur-Luciferase) as a potential differentiating tool for uterine fibroids which, unlike fluorescent proteins, do not require an external light source, but do require luciferin as the substrate for detecting the fluorescence-producing reporter gene as discussed, for example, in U.S. Pat. No. 9,790,562 incorporated herein by reference in the entirety. However, imaging tools employing Luciferases are not suitable for use in human subjects. Thus, there remains a need in the art for simplified, noninvasive systems that generate clinically significant and reliable results for differentiating malignant human subjects.

### SUMMARY

This disclosure describes compositions and methods for detecting uterine tumors.

As described below, in a first aspect the present disclosure provides a composition comprising an adenovirus Survivin-Sodium iodide symporter (Ad-SUR-NIS) vector comprising a Survivin promoter operatively linked to a sodium iodine symportor protein (NIS) protein-encoding sequence that is capable of infecting leiomyosarcoma (LMS) cells in vivo following systemic administration.

In embodiment of the first aspect the vector is configured to express NIS protein in a leiomyosarcoma (LMS), wherein in the presence of a radiotracer distinguishable imaging data is generated in LMS cells having sufficient energy emitted from the radiotracer that can be captured as imaging data from the LMS, and wherein only minimal energy is emitted from the radiotracer and captured as the imaging data from uterine fibroids (LM.)

In one embodiment of the first aspect, the radiotracer is I124.

In one embodiment of the first aspect the composition is for systemic administration.

In one embodiment of the first aspect the composition is configured to be administered according to a percentage Ad-SUR-NIS weight dose composition that induces NIS expression that allows for differential detection of a leiomyosarcoma (LMS).

In one embodiment of the first aspect provides a screening kit for noninvasively detecting a uterine sarcoma comprising the composition comprising an adenovirus Survivin-Sodium iodide symporter (Ad-SUR-NIS) vector stored in a suitable delivery vehicle, and optionally, a radiotracer configured for delivery after the composition.

In one embodiment the first aspect comprises one or more additional radiotracers

In one embodiment of the first aspect the screening data kit further comprises data access information configured to inform a therapeutic intervention based on the energy emitted and displayed in the imaging data collected upon use of the kit.

In a second aspect, the present disclosure provides a method of indirectly detecting uterine sarcoma in a subject, the method comprising: administering to the subject a composition comprising Ad-SUR-NIS and a radiotracer; exposing a target area of the subject where the presence of a sarcoma is suspected to PET-CT imaging; and capturing energy emitted from the target area of the subject in the imaging data, wherein the presence of the energy emitted correlates to symporter expression uptake of a radiotracer indirectly indicating the presence of a sarcoma in the target area of the subject.

In one embodiment of the second aspect, the step of exposing the target area comprises exposing the sarcoma to a selected weight percent of the composition selected from a uterine mass weight dose range and a radiotracer.

In one embodiment of the second aspect the administering step comprises administering the composition 0 hours to about 24 hours or more before radiotracer administration.

In one embodiment of the second aspect the capturing step comprises capturing energy emitted in imaging data using PET-CT imaging.

In a third aspect, the present disclosure provides a pre-surgical diagnostic system for detecting a leiomyosarcoma (LMS) based on survivin expression in uterine sarcoma of a subject comprising an (Ad-SUR-NIS) composition configured for delivery to an LMS in a weight dosage concentration selected from a uterine mass weight dose range; and the system configured for symporter expression in the LMS that can be detected in the presence of a radiotracer.

In one embodiment of the third aspect, the present disclosure provides a dosage configured to detect the presence of leiomyosarcoma (LMS) based on reporter gene expression of the radiotracer in PET-CT imaging data.

In a fourth aspect, the present disclosure provides a system for non-invasive detection of Ad-SUR-NIS activity levels in one or more uterine mass(es) in a subject comprising: a pre-imaging delivery composition configured to deliver a composition including Ad-SUR-NIS selected from a uterine mass weight dose range; and a radiotracer configured to emit energy based on gene expression of the Ad-SUR-NIS symporter and uptake of the radiotracer thereby; and an imaging device configured to capture energy signals emitted from the radiotracer as an indirect confirmation of the presence of a leiomyosarcoma (LMS).

In one embodiment of the fourth aspect the imaging device is further defined by a PET-CT device.

In a fifth aspect, the present disclosure provides a method of manufacturing a pre-surgical diagnostic system comprising the steps of preparing a gene-expressing adenovirus symporter vector encoding a Survivin promoter operatively linked to a sodium iodine symportor protein (NIS) protein-encoding sequence and configured for delivery to uterine tissue in a concentration based on weight and configured in accordance with a uterine mass weight dose range; filling a vector storage container with the vector; and packaging the storage container.

In one embodiment the fifth aspect, further comprises preparing one or more radiotracers; filling one or more additional storage containers with the one or more radiotracers; and packaging the one or more additional storage containers with the vector storage container.

In a sixth aspect, the present disclosure provides a composition of matter comprising: a viral vector containing a survivin promotor specifically configured for expression of a sodium iodine symportor protein (NIS), in a viral-mediated NIS gene delivery system.

In one embodiment of the sixth aspect, the composition is further defined by Ad-Sur-NIS.

In a seventh aspect, the present disclosure provides a viral platform containing a survivin promotor operatively linked to specifically express sodium iodine symportor (NIS) protein.

In one embodiment the seventh aspect the disclosure provides a method of administering the composition by systematic intravenous delivery of the composition and subsequent delivery of a radiotracer to generate distinguishable imaging between a cancerous and non-cancerous cell.

In one embodiment the seventh aspect the disclosure is further defined by a uterine mass weight dose range of a viral vector containing a survivin promotor configured to express NIS weight dose composition.

In one embodiment of the seventh aspect is a kit for noninvasively detecting a cancerous mass comprising: the composition stored in a suitable delivery vehicle, and optionally, a radiotracer configured for delivery after the composition.

In one embodiment the seventh aspect further comprises data access information configured to inform a next procedure based on the energy emitted and displayed in the imaging data collected upon use of the kit.

In an eighth aspect is a method of detecting sarcoma in a subject, the method comprising the steps of: administering a composition comprising a viral vector containing a survivin promotor configured to express a sodium iodine symportor protein (NIS) protein in the subject; delaying administration of a radiotracer for a delay time until the viral vector reaches a target area in the subject to express the NIS protein in the mass; administering the radiotracer after the delay time, where the radiotracer can be selected from Table 2; exposing the target area to PET-CT imaging to generate imaging data; and capturing energy emitted from the target area of the subject in the imaging data, wherein the presence of the energy emitted correlates to uptake of a radiotracer due to NIS expression indicating the presence of a cancerous mass in the target area of the subject.

In one embodiment the eighth aspect the step of exposing the target area comprises exposing the cancerous mass to a selected weight percent of the composition selected from a uterine mass weight dose range.

In one embodiment the eighth aspect administering step comprises administering the composition 8 hours to about 10 days before radiation exposure.

In one embodiment the eighth aspect the capturing step comprises capturing energy emitted in imaging data using PET-CT imaging.

In a ninth aspect is a pre-surgical diagnostic system for detecting survivin promoter activity in cancerous masses of a subject comprising: a viral vector containing a survivin promotor composition configured for delivery; and the system configured for uptake of a radiotracer due to sodium iodine symportor protein (NIS) expression.

In one embodiment the ninth aspect comprises a dosage of the viral vector configured to detect a cancerous mass based on uptake of a radiotracer due to NIS expression in PET-CT imaging data.

In a tenth aspect is a non-invasive detection of a cancerous mass using radiotracer in one or more subject mass(es) comprising: a pre-imaging delivery composition including a viral vector containing a survivin promotor specifically configured for expression of a sodium iodine symportor protein (NIS) protein.

In one embodiment the tenth aspect further comprises a radiotracer configured to emit energy based on expression of the NIS protein and uptake of the radiotracer thereby; and an imaging device configured to capture energy signals emitted from the radiotracer as a direct confirmation of the presence of tissue cancerous mass in the one or more subject masses.

In one embodiment the tenth aspect the imaging device is further defined by a PET-CT device.

In an eleventh aspect of the disclosure is a method of manufacturing a pre-surgical diagnostic system comprising the steps of preparing a viral vector containing a survivin promotor and sodium iodine symportor protein (NIS) protein configured for delivery to a target mass; filling a vector storage container with the vector; and packaging the storage container.

In one embodiment the eleventh aspect further comprising the step of preparing one or more radiotracers; filling one or more additional storage containers with the one or more radiotracers; and packaging the one or more additional storage containers with the vector storage container.

### BRIEF DESCRIPTION OF THE DRAWINGS

The accompanying drawings are included to provide a further understanding of the methods and compositions of the disclosure and are incorporated in and constitute a part of this specification. The drawings illustrate one or more embodiment(s) of the disclosure, and together with the description serve to explain the reagents and methods set forth in this disclosure.

FIG. 1 illustrates pre-clinical validation of data using exemplary compositions and methods as described herein.

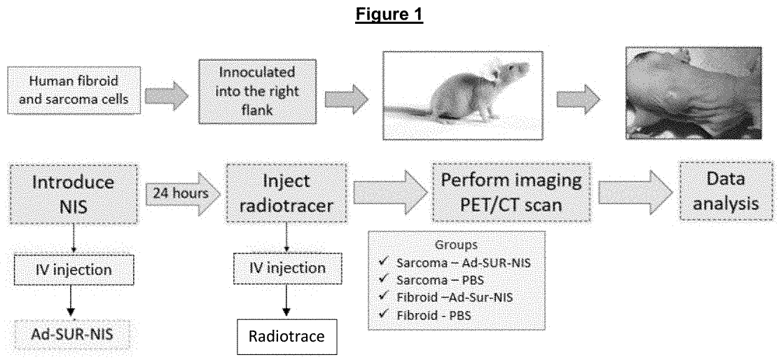

FIG. 2 illustrates (A) benign smooth muscle tumors originating from the myometrium, a condition that occurs in 77% of women. The presentation is abnormal uterine bleeding and pelvic pain with the treatment being medical and/or surgical. (B) Uterine sarcoma (3% of all uterine cancers) that are extremely aggressive tumors with a 5-year survival rate around 25%. The symptoms are similar to leiomyoma with the treatment involving surgery plus chemotherapy and/or radiation.

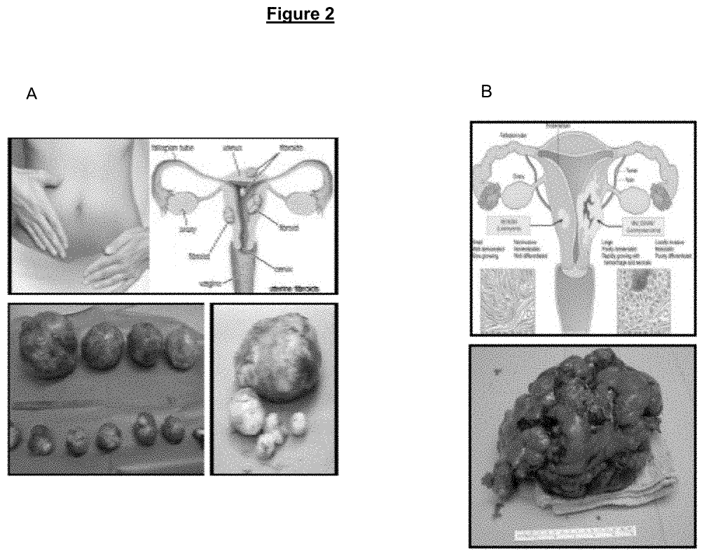

FIG. 3 illustrates a diagram of adenovirus-mediated NIS gene delivery. Ad-Sur-NIS is injected by a systemic route that will infect uterine sarcoma cells. The Ad-Sur-NIS enables NIS gene expression in such cells and NIS protein is displayed on the cell membrane in infected cells. Radiotracer administered systemically s taken-up in NIS-expressing tumor cells and can be detected.

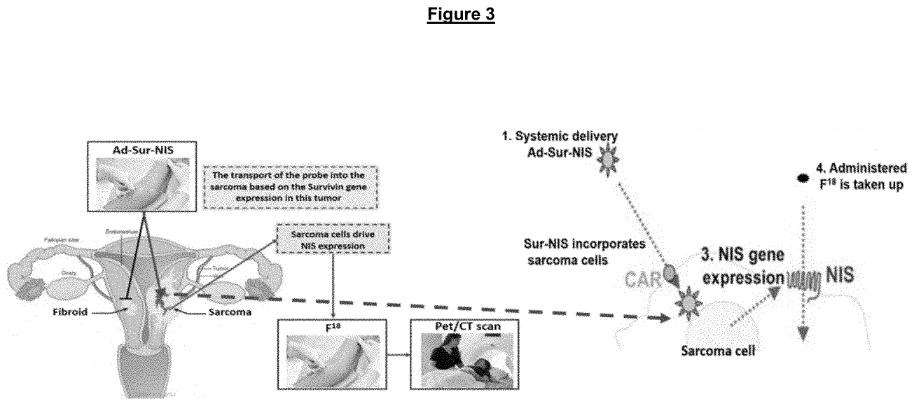

FIG. 4 illustrates the promoter of the Survivin gene is more highly expressed in uterine sarcomas, but not in fibroids. The Ad_SUR_NIS presence is able to identify and bind the promoter of the Sur in LMS and start the expression of NIS. The NIS expression is detected during PET/CT scan after the administration of F18.

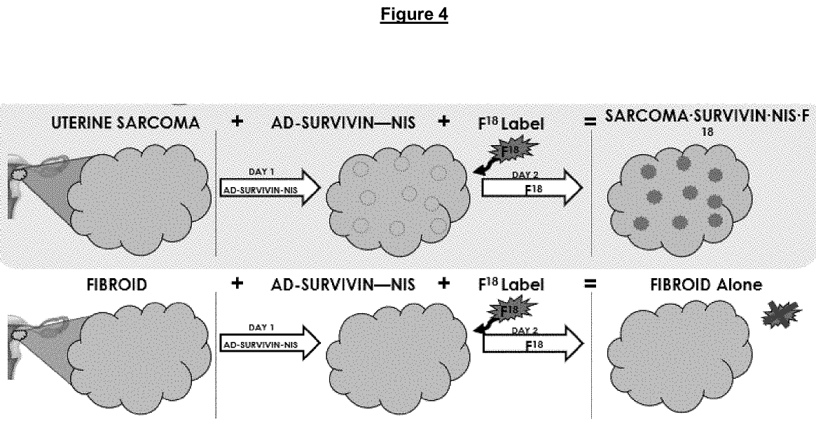

FIG. 5 illustrates an in vitro analysis of Ad-Sur-NIS activity in uterine myometrium, fibroid and sarcoma cells lines. Cells were transfected with Ad-Sur-NIS followed with I¹²⁴ incubation and PET scan. Higher expression of NIS in sarcomas cells was detected after the PET scan, illustrating that Ad-Sur-NIS is selective for uterine sarcoma cells.

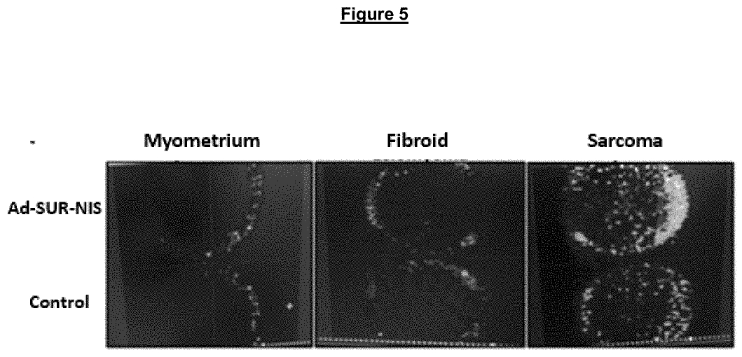

FIG. 6 illustrates the experimental design of in vivo experiments and PET-CT Scan.

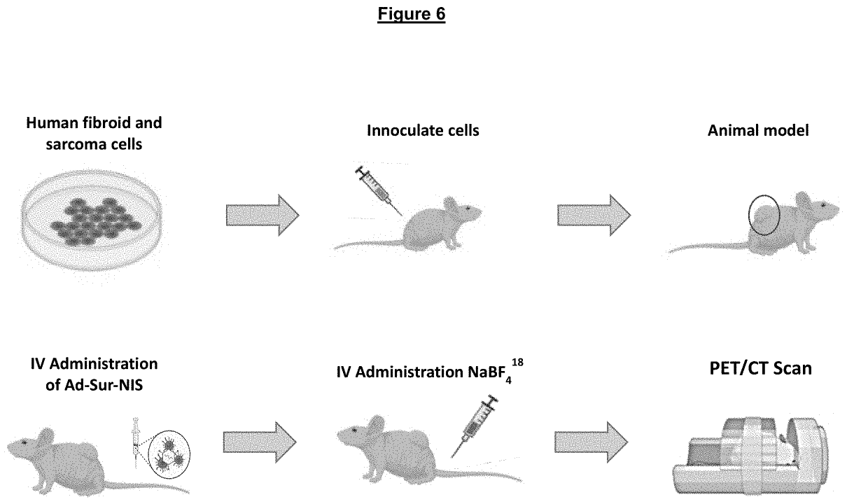

FIG. 7 illustrates a diagram of an in vivo experiment. Fibroid and sarcoma animal models were created, and after 7 days the tumors were observed to have developed. Ad-Sur-NIS were injected systemically and after 24 hours the radiotracer I¹²⁴ was administrated systemically followed by PET/CT scan.

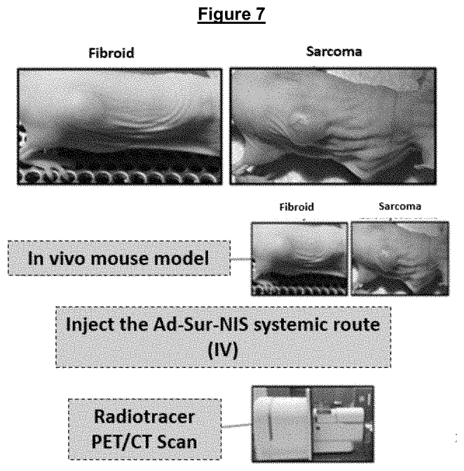

FIG. 8 illustrates a PET/CT scan in the animals after I¹²⁴ radiotracer injection. (A) Ad-Sur-NIS is able to differentiate uterine sarcoma from fibroid, whereas (B) shows high activity of the NIS reporter.

FIG. 9 illustrates Ad-Sur-NIS kinetics in animals. (A) Increased radiotracer uptake attributable to Ad-Sur-NIS in the LMS tumors when compared to LM. Circles indicates statistically significant differences. (B) Illustrates decrease in decay in LMS Ad-Sur-NIS compared to LM. Circle indicates statistically significant differences.

FIG. 10 illustrates PET/CT scan images from the animals LMS AD-SUR-NIS, LM AD-SUR-NIS, LMS PBS, and LM PBS showing the higher expression of NIS in LMS AD-SUR-NIS compare to LM AD-SUR-NIS after F18 administration. The yellow circles are identified the tumors.

FIG. 11 illustrates liver biomarker results in LMS AD-SUR-NIS, LM AD-SUR-NIS, LMS PBS, and LM PBS mice.

FIG. 12 illustrates safety of AD-SUR-NIS as demonstrated by liver and blood indices.

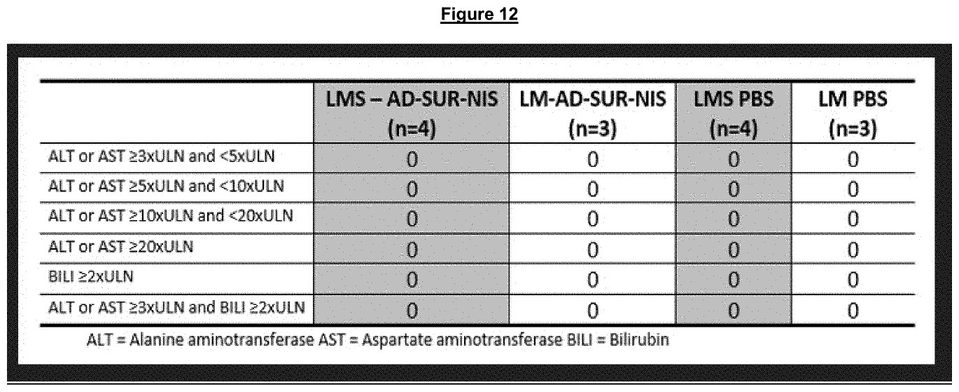

FIG. 13 illustrates a summary of histopathologic evaluations.

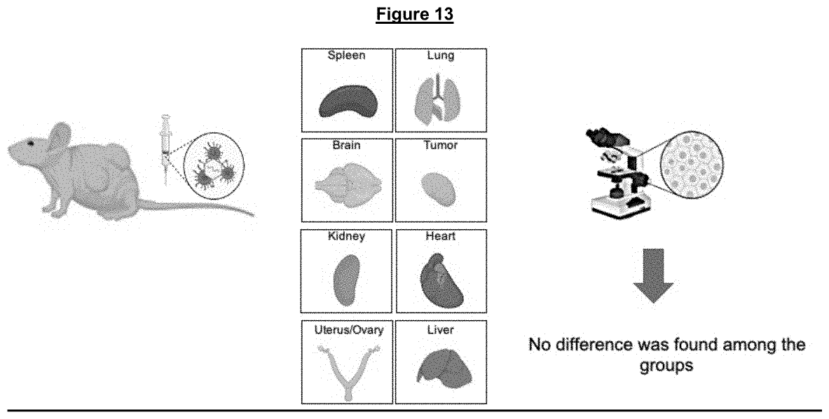

FIG. 14 illustrates histopathologic results in PBS and AD-SUR-NIS mice in brain, heart, kidney, and liver. Magnification at 20×.

FIG. 15 illustrates histopathologic results in PBS and AD-SUR-NIS mice in lung, ovary, spleen, and uterus. Magnification at 20×.

### DETAILED DESCRIPTION OF PREFERRED EMBODIMENTS

Provided herein are methods, kits, systems, and compositions for detection of benign versus malignant uterine masses.

Unless defined otherwise, all technical and scientific terms used herein have the meaning commonly understood by a person skilled in the art to which the claimed invention set forth herein belongs. The terminology used herein is for describing particular embodiments only and is not intended to be limiting of the claimed invention set forth herein. All technical and scientific terms used herein have the same meaning.

The following terms may have meanings ascribed to them below, unless specified otherwise. However, it should be understood that other meanings known or understood by those having ordinary skill in the art are also within the scope of the claimed invention set forth herein. All publications, patent applications, patents, and other references mentioned or discussed herein are expressly incorporated by reference in their entireties. In the case of conflict, the present specification, including definitions, will control. In addition, the materials, methods, and examples are illustrative only and not intended to be limiting.

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps.

As used herein, the singular forms “a,” “an,” and “the” include plural referents unless the context clearly indicates otherwise.

As used herein, the term “or” means, and is used interchangeably with, the term “and/or,” unless context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

In accordance with the invention set forth herein, gene-based molecular bio-imaging probes, and methods of their uses, for non-invasive differentiation between benign human uterine fibroids and uterine sarcoma in women with suspicious uterine masses is set forth. Bio-imaging probes constructed in accordance with the invention set forth herein provide improved medical practice standards of care, and enable effective triage of patients with suspicious uterine mass(es), which can significantly impact women's health worldwide, while also providing a reduction in the number of surgeries performed in accordance with current standards. In addition, prompt early diagnosis and treatment in accordance with the invention set forth herein set forth herein can lead to a better outcome/survival for sarcoma patients.

Exemplary embodiments constructed in accordance with the invention set forth herein disclosed herein can comprise a composition of matter including a viral vector containing a survivin promotor specifically configured for expression of a sodium iodine symportor protein (NIS). In accordance with the invention set forth herein, such composition(s) can be captured as Ad-SUR-NIS to represent one embodiment of an adenovirus-mediated NIS gene delivery. Ad-Sur-NIS can be used to infect sarcoma cells, and particularly uterine sarcoma cells, by systemic injection. In the infected cells Ad-Sur-NIS can enable NIS gene expression, and NIS protein can be displayed on the cell membrane in infected cells. A radiotracer can be administered systemically and is taken up in NIS-expressing tumor cells.

Some exemplary embodiments constructed in accordance with the invention set forth herein set forth herein can provide adenovirus survivin-sodium iodide symporter (Ad-Sur-Nis) systems as gene-based bioimaging tools that can generate imaging data containing reporter expression uptake of a suitable radiotracer, indicative of survivin expression, to differentiate between uterine fibroid and sarcoma in women with suspicious uterine masses without surgical intervention. For example, some exemplary embodiments can be configured to generate imagining data containing symporter uptake information from a suitable radioisotope, such as Fluorine-18 (F18). All embodiments set forth in accordance with the invention set forth herein provide luciferase-free differentiating systems for collecting energy emission of radiotracer(s) in imaging data indicating uterine sarcoma. Since LM has minimal expression of survivin gene and thus do not have activated reporter gene expression using the reagents and methods disclosed herein, an accurate pre-surgical diagnosis can be achieved based on the imaging data wherein radiotracer emissions are specific for uterine sarcoma.

In accordance with the invention set forth herein, exemplary compositions can comprise: Adenovirus Survivin-Sodium iodide symporter (Ad-SUR-NIS) configured to specifically express the NIS protein in leiomyosarcoma (LMS) due to the increased presence of the survivin protein which acts as a promotor and to generate distinguishable imaging data based on significant energy emitted from the radiotracer and captured in the imaging data for LMS, versus minimal energy emitted from the radiotracer and captured in the imaging data for leiomyoma (LM) due to the fact that LM lacks significant (if any) survivin protein expression. The composition can be further defined by a suitable weight-based dose, such as dosages standard for delivery of adenovirus delivery particles. For example, for LMS detection, a suitable dose can be in the range of 1×10⁹ to 4×10¹¹% Ad-SUR-NIS weight dose composition (hereinafter referred to as “uterine mass weight dose range”), where an exemplary dose of 1-4×10¹⁰ PFU/KG % Ad-SUR-NIS weight dose composition is considered suitable for the average weight adult woman patient in the United States. As disclosed herein, the composition contains the vector. One of skill in the art will further understand that composition-containing vector may further require necessary components. The composition can further comprise one or more additional radiotracers. The one or more radiotracer provides the ability to detect differential expression of NIS due to expression of NIS in

The radiotracers described herein are advantageous over previous methods and compositions as the (reduced) amounts of radiotracer required for detection of malignant uterine masses mitigates potential harm to the patient but allows detection of malignant masses. As such the compositions and associated methods disclosed herein overcomes the limitations of previous tracers, including luciferase.

A screening kit for noninvasively detecting a malignant uterine sarcoma can include an Ad-SUR-NIS composition stored in a suitable delivery vehicle, configured for delivery after the Ad-SUR-NIS composition. A suitable radiotracer, such as F18 or other suitable tracer, can be configured for delivery after the Ad-SUR-NIS composition (see Table 2 below). The screening kit can further comprise data access information configured to inform a procedure based on the energy emitted and displayed in the imaging data collected upon use of the screening kit.

An exemplary method of indirectly detecting uterine sarcoma cells in a subject is set forth. The method can include the steps of: a) administering to the subject a composition comprising Ad-SUR-NIS and, concomitantly or thereafter a radiotracer; b) exposing a target area of the subject where the presence of a sarcoma is suspected to PET-CT imaging; and c) capturing energy emitted from the target area of the subject in the imaging data, wherein the presence of the energy emitted from the radiotracer correlates to symporter expression, indicating the presence of a sarcoma in the target area of the subject. One of skill in the art will understand that in a further exemplary method an alternate imaging method could be used.

Another exemplary method of detecting sarcoma cells in a subject is set forth. The method can include the steps of: a) administering to the subject a composition comprising Ad-SUR-NIS; b) waiting a sufficient time for cancerous cells to express the NIS protein; c) administering a radiotracer; d) exposing a target area of the subject where the presence of a sarcoma is suspected to PET-CT imaging; and e) capturing energy emitted from the target area of the subject in the imaging data, wherein the presence of the energy emitted correlates to symporter expression from the radiotracer indicating the presence of a sarcoma in the target area of the subject.

If desired, the method can further include the step of exposing the target area to include exposing the cells to a weight percent of the composition from the uterine mass weight dose range and a radiotracer comprising a suitable isotope, such as an isotope set forth in Table 1, or similar. Further, the administering step can include administering the composition 0 hours to about 10 days or more before radiotracer administration. The capturing step can include capturing energy emitted in imaging data using PET-CT imaging.

A pre-surgical diagnostic system for detecting survivin promoter activity in uterine tissue of a subject is also set forth. The system can include an Ad-SUR-NIS composition configured for delivery in a suitable dosage. For LMS detection, for example, a weight dosage concentration can be selected from the uterine mass weight dose range in most instances, and configured for delivery followed by a suitable radiotracer, such as F18. The weight dosage of the system can be configured to produce a level of survivin promoter in order to express the NIS protein sufficiently to clearly capture the energy emitted due to the uptake of the radiotracer, wherein the energy emitted is indicative of sarcoma in promoter-activated tissue in the PET-CT imaging data.

A system for non-invasive detection of Ad-SUR-NIS activity levels in subject tumors, such as uterine masses, is set forth. The system can include a pre-imaging delivery composition configured to deliver a composition including Ad-SUR-NIS in a suitable concentration; a radiotracer configured to emit energy based on gene expression of symporter protein and uptake of the radiotracer, and an imaging device configured to capture energy signals emitted from the radiotracer as an indirect confirmation of the presence of cancerous tissue. The imaging device can be further defined as a PET-CT device.

A method of manufacturing a pre-surgical uterine or other mass diagnostic system can include the steps of preparing a gene-expressing adenovirus symporter vector configured for delivery to the uterus or other location in a suitable concentration, such as a concentration based on weight; filling a vector storage container with the vector; and packaging the storage container. The method can further include the step of preparing one or more radiotracers, filling one or more additional storage containers with the one or more radiotracers, and packaging the one or more additional storage containers with the vector storage container.

In accordance with the invention set forth herein disclosed herein, the reagents and methods of this invention set forth herein can successfully detect human uterine sarcoma as shown in animal model studies, having a 100% success for detecting human sarcoma in the animal model. Image data and datasets can be configured to optimize the image protocol for early versus late capture of sarcomas, in accordance with the invention set forth herein. Such datasets can include algorithms for determining go/no-go success criteria for treating malignant masses detected via the system herein.

In accordance with the invention set forth herein set forth herein, delivery of an adenovirus to uterine masses can be achieved via suitable method, such as IV Injection or other suitable delivery method. Once the virus reaches the target tissue, a suitable radiotracer, such as F18, can be administered to detect surviving-dependent NIS expression.

The invention set forth herein set forth herein is advantageous compared with current radiologic imaging modalities, including ultrasound, MRI, and positron emission tomography/computed tomography that are not able to differentiate between LMS vs LM. Use of an Adenovirus Survivin-Sodium iodide symporter (Ad-Sur-NIS) system for differentiating between LM and LMS provides significant value in the early diagnosis of patients with LMS, and subsequently improves prognosis and treatment outcome. The recombinant adenovirus Ad-Sur-NIS, uses the survivin promoter to drive NIS expression

Genes expressed via the surviving promoter show selectively higher activity in LMS. As set forth herein this increased expression has been used to provide a method for detecting LMS by systemic administration of a vector that utilizes a survivin promoter to express a suitable reporter gene. Using NIS as the reporter gene in the systems discloses herein, LMS-specific expression can be detected in the presence of a radiotracer using PET-CT scan. Detection can provide a differential diagnosis of LMS over LM, due to minimum expression of the reporter gene in LM, as illustrated in the imaging data shown below.

Further, systems constructed in accordance with the invention set forth herein set forth herein can also monitor the effectiveness of treatment, and/or provide a prognostic tool choice of the most appropriate surgical procedure. For example, systems according to the invention set forth herein set forth herein can be configured to inform an indication for minimally invasive procedure (morcellation) versus open surgery, with faster recurve and major costs savings. Further, systems constructed in accordance with the invention set forth herein can confirm benign tissue, thus avoiding surgical intervention for uterine fibroids.

Adenovirus vectors are the most commonly employed vectors for cancer gene therapy. They are also used for gene therapy and as vaccines to express foreign antigens. It can be replication-defective; certain essential viral genes are deleted and replaced by a cassette that expresses a foreign therapeutic gene. More than 400 gene therapy trials have been or are being conducted with human Ad vectors. Most of these trials are for the treatment of cancer.

Compositions constructed in accordance with the invention set forth herein can contain suitable percentages of SEQ ID NO. 1, as shown in Table 1, of an exemplary Ad-hSurvivin-hSLC5A5 vector, to achieve a diagnostic output. Other similar vectors are contemplated herein as well

TABLE 1
Ad-hSurvivin-hSLC5A5 Vector

| | |
|---|---|
| SEQ ID | 1 CTTTCCTGCG TTATCCCCTG ATTCTGTGGA TAACCGTATT ACCGCTAGCA |
| NO. 1 | 51 TGGATCTCGG GGACGTCTAA CTACTAAGCG AGAGTAGGGA ACTGCCAGGC |
| | 101 ATCAAATAAA ACGAAAGGCT CAGTCGGAAG ACTGGGCCTT TCGTTTTATC |
| | 151 TGTTGTTTGT CGGTGAACGC TCTCCTGAGT AGGACAAATC CGCCGGGAGC |
| | 201 GGATTTGAAC GTTGTGAAGC AACGGCCCGG AGGGTGGCGG GCAGGACGCC |
| | 251 CGCCATAAAC TGCCAGGCAT CAAACTAAGC AGAAGGCCAT CCTGACGGAT |
| | 301 GGCCTTTTTG CGTTTCTACA AACTCTTCCT GTTAGTTAGT TACTTAAGCT |
| | 351 CGGGCCCCAA ATAATGATTT TATTTTGACT GATAGTGACC TGTTCGTTGC |
| | 401 AACAAATTGA TAAGCAATGC TTTTTTATAA TGCCAACTTT GTACAACAAA |
| | 451 GCAGGCTTTA AAGGAACCAA TTCAGTCGAC GGCAGGGACG AGCTGGCGCG |
| | 501 GCGTCGCTGG GTGCACCGCG ACCACGGGCA GAGCCACGCG GCGGGAGGAC |
| | 551 TACAACTCCC GGCACACCCC GCGCCGCCCC GCCTCTACTC CCAGAAGGCC |
| | 601 GCGGGGGGTG GACCGCCTAA GAGGGCGTGC GCTCCCGACA TGCCCCGCGG |
| | 651 CGCGCCATTA ACCGCCAGAT TTGAATCGCG GGACCCGTTG GCAGAGGTGG |
| | 701 CGGCGGCGGC ATGGGTGCCC CGACGTTGCC CCCTGCCTGG AAGCTTGGAT |
| | 751 CCCCACCATG GAGGCCGTGG AGACCGGGGA ACGGCCCACC TTCGGAGCCT |
| | 801 GGGACTACGG GGTCTTTGCC CTCATGCTCC TGGTGTCCAC TGGCATCGGG |
| | 851 CTGTGGGTCG GGCTGGCTCG GGGCGGGCAG CGCAGCGCTG AGGACTTCTT |
| | 901 CACCGGGGGC CGGCGCCTGG CGGCCCTGCC CGTGGGCCTG TCGCTGTCTG |
| | 951 CCAGCTTCAT GTCGGCCGTG CAGGTGCTGG GCGTGCCGTC GGAGGCCTAT |
| | 1001 CGCTATGGCC TCAAGTTCCT CTGGATGTGC CTGGGCCAGC TTCTGAACTC |
| | 1051 GGTCCTCACC GCCCTGCTCT TCATGCCCGT CTTCTACCGC CTGGGCCTCA |
| | 1101 CCAGCACCTA CGAGTACCTG GAGATGCGCT TCAGCCGCGC AGTGCGGCTC |
| | 1151 TGCGGGACTT TGCAGTACAT TGTAGCCACG ATGCTGTACA CCGGCATCGT |
| | 1201 AATCTACGCA CCGGCCCTCA TCCTGAACCA AGTGACCGGG CTGGACATCT |
| | 1251 GGGCGTCGCT CCTGTCCACC GGAATTATCT GCACCTTCTA CACGGCTGTG |
| | 1301 GGCGGCATGA AGGCTGTGGT CTGGACTGAT GTGTTCCAGG TCGTGGTGAT |
| | 1351 GCTAAGTGGC TTCTGGGTTG TCCTGGCACG CGGTGTCATG CTTGTGGGCG |
| | 1401 GGCCCCGCCA GGTACTCACG CTGGCCCAGA ACCACTCCCG GATCAACCTC |
| | 1451 ATGGACTTTA ACCCTGACCC GAGGAGCCGC TATACATTCT GGACTTTTGT |
| | 1501 GGTGGGTGGC ACGTTGGTGT GGCTCTCCAT GTATGGCGTG AACCAGGCGC |
| | 1551 AGGTGCAGCG CTACGTGGCT TGCCGCACAG AGAAGCAGGC CAAGCTGGCC |
| | 1601 CTGCTCATCA ACCAGGTCGG CCTGTTCCTG ATCGTGTCCA GCGCTGCCTG |
| | 1651 CTGTGGCATC GTCATGTTTG TGTTCTACAC TGACTGCGAC CCTCTCCTCC |
| | 1701 TGGGGCGCAT CTCTGCCCCA GACCAGTACA TGCCTCTGCT GGTGCTGGAC |
| | 1751 ATCTTCGAAG ATCTGCCTGG AGTCCCCGGG CTTTTCCTGG CCTGTGCTTA |
| | 1801 CAGTGGCACC CTCAGCACAG CATCCACCAG CATCAATGCT ATGGCTGCAG |
| | 1851 TCACTGTAGA AGACCTCATC AAACCTCGGC TGCGGAGCCT GGCACCCAGG |
| | 1901 AAACTCGTGA TTATCTCCAA GGGGCTCTCA CTCATCTACG GATCGGCCTG |
| | 1951 TCTCACCGTG GCAGCCCTGT CCTCACTGCT CGGAGGAGGT GTCCTTCAGG |
| | 2001 GCTCCTTCAC CGTCATGGGA GTCATCAGCG GCCCCCTGCT GGGAGCCTTC |
| | 2051 ATCTTGGGAA TGTTCCTGCC GGCCTGCAAC ACACCGGGCG TCCTCGCGGG |
| | 2101 ACTAGGCGCG GGCTTGGCGC TGTCGCTGTG GGTGGCCTTG GGCGCCACGC |
| | 2151 TGTACCCACC CAGCGAGCAG ACCATGAGGG TCCTGCCATC GTCGGCTGCC |
| | 2201 CGCTGCGTGG CTCTCTCAGT CAACGCCTCT GGCCTCCTGG ACCCGGCTCT |
| | 2251 CCTCCCTGCT AACGACTCCA GCAGGGCCCC CAGCTCAGGA ATGGACGCCA |
| | 2301 GCCGACCCGC CTTAGCTGAC AGCTTCTATG CCATCTCCTA TCTCTATTAC |
| | 2351 GGTGCCCTGG GCACGCTGAC CACTGTGCTG TGCGGAGCCC TCATCAGCTG |
| | 2401 CCTGACAGGC CCCACCAAGC GCAGCACCCT GGCCCCGGGA TTGTTGTGGT |
| | 2451 GGGACCTCGC ACGGCAGACA GCATCAGTGG CCCCCAAGGA AGAAGTGGCC |
| | 2501 ATCCTGGATG ACAACTTGGT CAAGGGTCCT GAAGAACTCC CCACTGGAAA |
| | 2551 CAAGAAGCCC CCTGGCTTCC TGCCCACCAA TGAGGATCGT CTGTTTTTCT |
| | 2601 TGGGGCAGAA GGAGCTGGAG GGGGCTGGCT CTTGGACCCC CTGTGTTGGA |
| | 2651 CATGATGGTG GTCGAGACCA GCAGGAGACA AACCTCTGAG AATTCTGCAG |
| | 2701 ATATCCAGCA CAGTGGCGGC CGCTCGAGTC TAGAGGGCCC TTCGAAGGTA |
| | 2751 AGCCTATCCC TAACCCTCTC CTCGGTCTCG ATTCTACGCG TACCGGTCAT |
| | 2801 CATCACCATC ACCATTGAGT TTAAACCCGC TGATCAGCCT CGACTGTGCC |
| | 2851 TTCTAGTTGC CAGCCATCTG TTGTTTGCCC CTCCCCCGTG CCTTCCTTGA |
| | 2901 CCCTGGAAGG TGCCACTCCC ACTGTCCTTT CCTAATAAAA TGAGGAAATT |
| | 2951 GCATCGCATT GTCTGAGTAG GTGTCATTCT ATTCTGGGGG GTGGGGTGGG |
| | 3001 GCAGGACAGC AAGGGGGAGG ATTGGGAAGA CAATAGCAGG CATGCTGGGG |
| | 3051 ATGCGGTGGG CTCTATGGCT TCTGAGGCGG AAAGAACCAG ATCTAGACCC |
| | 3101 AGCTTTCTTG TACAAAGTTG GCATTATAAG AAAGCATTGC TTATCAATTT |
| | 3151 GTTGCAACGA ACAGGTCACT ATCAGTCAAA ATAAAATCAT TATTTGCCAT |
| | 3201 CCAGCTGCAG CTCTGGCCCG TGTCTCAAAA TCTCTGATGT TACATTGCAC |
| | 3251 AAGATAAAAA TATATCATCA TGAACAATAA AACTGTCTGC TTACATAAAC |
| | 3301 AGTAATACAA GGGGTGTTAT GAGCCATATT CAACGGGAAA CGTCGAGGCC |
| | 3351 GCGATTAAAT TCCAACATGG ATGCTGATTT ATATGGGTAT AAATGGGCTC |
| | 3401 GCGATAATGT CGGGCAATCA GGTGCGACAA TCTATCGCTT GTATGGGAAG |
| | 3451 CCCGATGCGC CAGAGTTGTT TCTGAAACAT GGCAAAGGTA GCGTTGCCAA |
| | 3501 TGATGTTACA GATGAGATGG TCAGACTAAA CTGGCTGACG GAATTTATGC |
| | 3551 CTCTTCCGAC CATCAAGCAT TTTATCCGTA CTCCTGATGA TGCATGGTTA |
| | 3601 CTCACCACTG CGATCCCCGG AAAAACAGCA TTCCAGGTAT TAGAAGAATA |
| | 3651 TCCTGATTCA GGTGAAAATA TTGTTGATGC GCTGGCAGTG TCCCTGCGCC |
| | 3701 GGTTGCATTC GATTCCTGTT TGTAATTGTC CTTTTAACAG CGATCGCGTA |
| | 3751 TTTCGTCTCG CTCAGGCGCA ATCACGAATG AATAACGGTT TGGTTGATGC |
| | 3801 GAGTGATTTT GATGACGAGC GTAATGGCTG GCCTGTTGAA CAAGTCTGGA |
| | 3851 AAGAAATGCA TAAACTTTTG CCATTCTCAC CGGATTCAGT CGTCACTCAT |
| | 3901 GGTGATTTCT CACTTGATAA CCTTATTTTT GACGAGGGGA AATTAATAGG |
| | 3951 TTGTATTGAT GTTGGACGAG TCGGAATCGC AGACCGATAC CAGGATCTTG |
| | 4001 CCATCCTATG GAACTGCCTC GGTGAGTTTT CTCCTTCATT ACAGAAACGG |
| | 4051 CTTTTTCAAA AATATGGTAT TGATAATCCT GATATGAATA AATTGCAGTT |
| | 4101 TCATTTGATG CTCGATGAGT TTTTCTAATC AGAATTGGTT AATTGGTTGT |
| | 4151 AACATTATTC AGATTGGGCC CCGTTCCACT GAGCGTCAGA CCCGGTAGAA |
| | 4201 AAGATCAAAG GATCTTCTTG AGATCCTTTT TTTCTGCGCG TAATCTGCTG |
| | 4251 CTTGCAAACA AAAAAACCAC CGCTACCAGC GGTGGTTTGT TTGCCGGATC |
| | 4301 AAGAGCTACC AACTCTTTTT CCGAAGGTAA CTGGCTTCAG CAGAGCGCAG |
| | 4351 ATACCAAATA CTGTTCTTCT AGTGTAGCCG TAGTTAGGCC ACCACTTCAA |
| | 4401 GAACTCTGTA GCACCGCCTA CATACCTCGC TCTGCTAATC CTGTTACCAG |
| | 4451 TGGCTGCTGC CAGTGGCGAT AAGTCGTGTC TTACCGGGTT GGACTCAAGA |
| | 4501 CGATAGTTAC CGGATAAGGC GCAGCGGTCG GGCTGAACGG GGGGTTCGTG |
| | 4551 CACACAGCCC AGCTTGGAGC GAACGACCTA CACCGAACTG AGATACCTAC |
| | 4601 AGCGTGAGCT ATGAGAAAGC GCCACGCTTC CCGAAGGGAG AAAGGCGGAC |
| | 4651 AGGTATCCGG TAAGCGGCAG GGTCGGAACA GGAGAGCGCA CGAGGGAGCT |
| | 4701 TCCAGGGGGA AACGCCTGGT ATCTTTATAG TCCTGTCGGG TTTCGCCACC |
| | 4751 TCTGACTTGA GCGTCGATTT TTGTGATGCT CGTCAGGGGG GCGGAGCCTA |
| | 4801 TGGAAAAACG CCAGCAACGC GGCCTTTTTA CGGTTCCTGG CCTTTTGCTG |
| | 4851 GCCTTTTGCT CACATGTT |

Suitable radiotracers can include, but is not limited to the tracers provided in Table 2.

TABLE 2

| Primary | | | | | | Absorbed | Clinically |
|---|---|---|---|---|---|---|---|
| Use | Tracer | Half-Life | | NIS Affinity | Sensitivity | Rad. Dose | Approved |
| PET | ¹⁸F-BF⁴ | 110 | min | ++ | +++ | + | In Trials |
| Imaging | ¹²⁴I | 4.2 | days | ++ | + | ++ | In Trials |
| | ¹⁸F-SO₃F | 110 | min | +++ | +++ | + | No |
| SPECT | ^{99m}TC-pertecenetate | 6 | h | +++ | ++ | + | Yes |
| Imaging | ¹²³I | 13.2 | h | ++ | + | ++ | Yes |

Strong survivin expression is observed in the vast majority of cancers. Survivin is strongly expressed in LMS and is thus a target as described herein.

The use of diagnostic compositions and methods constructed in accordance with the invention set forth herein can be extended to differentiate between cancerous and non-cancerous cells, such as fibroids, polyps and/or cysts in all of the cancer types above.

In addition to adenovirus vectors, additional viral vector platforms may include: Herpes simplex viruses, coxsackieviruses, lentiviruses, leukemia viruses, retroviruses, measles, vaccinia, reoviruses, parvoviruses, polio/rhinoviruses, vesicular stomatitis virus, for example, in accordance with the invention set forth herein.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### EXAMPLES

### Materials and Methods

### Human Fibroid and Human Uterine Leiomyosarcoma Cells

An immortalized human fibroid cell line (LM) was cultured and maintained in phenol red-free, 10% fetal bovine serum Dulbecco's Modified Eagle Medium: Nutrient Mixture F-12. A human leiomyosarcoma (LMS) cell line (SK-UT1, ATCC HTB-114TM) (ATCC, Manassas, Va., USA) was cultured and maintained in ATCC-formulated Eagle's Minimum Essential Medium with 10% of fetal bovine serum.

### Reagents

Ad-Sur-NIS was produced by Vector Biolabs. The PET imaging tracer, F18-labeled sodium tetrafluoroborate (F18-NaBF4), was purchased from the Cyclotron Facility at the University of Chicago.

### Animal Model

Fifty-four (54) nu/nu nude mice were purchased from Charles River. The mice were handled according to an approved protocol (18-174) and all the mice were maintained in 12 h light/dark cycle and provide with water as standard diet ad libitum in a pathogen-free facility under climate-controlled. 2×10⁷ of human leiomyosarcoma cells or human fibroid cells were inoculated into the right flank with 1:1 Matrigel and fetal bovine serum (FBS). After tumor development, animals were randomized separately in groups, designated LMS Ad-Sur-NIS, LMS PBS, LM Ad-Sur-NIS and LM PBS.

### PET/CT Scan

Forty (40) animals (ten apiece of LMS Ad-Sur-NIS, LMS PBS, LM Ad-Sur-NIS, and LM PBS) were used to produce PET/CT images using a micro PET/CT scanner (Trans-PET Discoverist 80, Raycan Technology Co., Ltd., Suzhou, China). 24 hours before the PET/CT scan, the animals received 1×10⁹ PFU/0.2 ml/mouse or PBS through retro-orbital injection. On the day of PET/CT scan, a dose of 300-400 uCi of F18-NaBF₄ was injected into each mouse through the tail vein. Then each mouse was put on the sample stage of the PET scanner with a heating pad and under isoflurane anesthesia. Two PET/CT scans were conducted for each mouse, at min 5 and 45 minutes after the injection. The mouse was taken out of the scanner for rest between the two scans. Each PET scan lasted for 10 minutes in static mode. PET and CT images were reconstructed using the software PiSYS provided with the scanner and were exported in DICOM format for analysis.

### PET/CT Scan Analysis

Carimas 2.10 (Turku PET Centre, Turku, Finland; http://www.turkupetcentre.fi/carimas) was used to analyze PET images. A 3-dimensional region of interest (ROI) was drawn for the tumor area in each mouse. Corresponding CT images were overlaid with the PET images as an anatomical reference. The ROI was smoothed once before the PET intensities (i.e., the F18 activities) were exported. The standardized uptake value (SUVmax or SUVmean) for each ROI was calculated by using the formula, SUV=A/ρ/D/W, where, A is the maximum or mean F18 activity of the ROI, ρ is the density of the mouse (˜1 g/ml), D is the total injected dose, and W is the weight of the mouse. SUVmax values were used to create the graphics.

### Safety Study—H&E Stain and Metabolic Panel

Fourteen (14) animals were used to determine the safety of Ad-Sur-NIS (LMS Ad-Sur-NIS n=4, LMS PBS n=4, LM Ad-Sur-NIS n=3, LM PBS n=3). 24 hours after Ad-Sur-NIS injection, blood (serum) and organs (brain, kidney, liver, lung, heart, ovary, uterus spleen and tumor) were collected from each animal. Serum from each animal was used to evaluate their liver function, a chemical blood panel were performed, and organs fixed in 10% buffered formalin for 24 h, then embedded with paraffin and subjected to H&E stain. H&E slides were evaluated by a veterinary pathologist.

### Statistical Analysis

Comparison of 2 groups was carried out using student t-test for parametric distribution and Mann Whitney test for nonparametric distribution. Comparison of multiple groups was carried out by analysis of variance (ANOVA) followed by a post-test using Tukey for parametric distribution and Kruskal-Wallis test followed by a post-test Dunns for nonparametric distribution, using GraphPad Prism 5 Software. Data were presented as mean±standard error (SE). The significant difference was defined as p<0.05.

### Results

Successful use of the compositions and methods disclosed herein is illustrated in the figures, which show administration of Ad-Sur-NIS accompanied by radioiodine administration and differential detection of LMS in vivo.

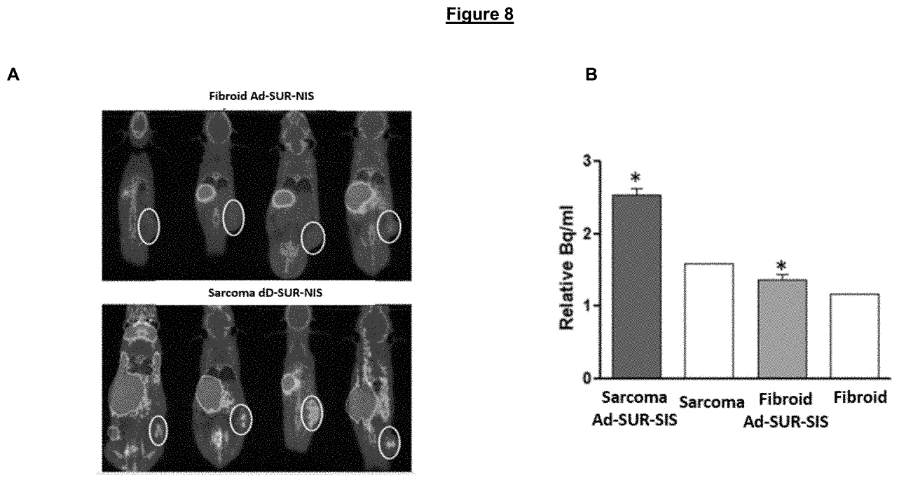

PET/CT scan in animals after the radiotracer, I¹²⁴ demonstrates that the Ad-Sur-NIS is able to differentiate the uterine sarcoma from the fibroid, with high activity of the NIS reporter (FIG. 8, shaded bars).

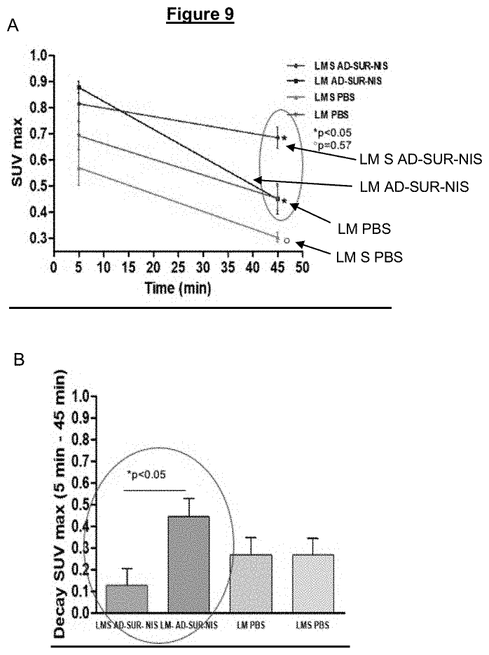

Ad-Sur-NIS kinetics in animals demonstrates significantly increased radiotracer uptake attributable to Ad-Sur-NIS in the LMS tumors when compared to LM (FIG. 9, Panel A circled areas). A statistically significant decrease in decay in LMS Ad-Sur-NIS was observed compared to LM (FIG. 9, Panel B, Circled area).

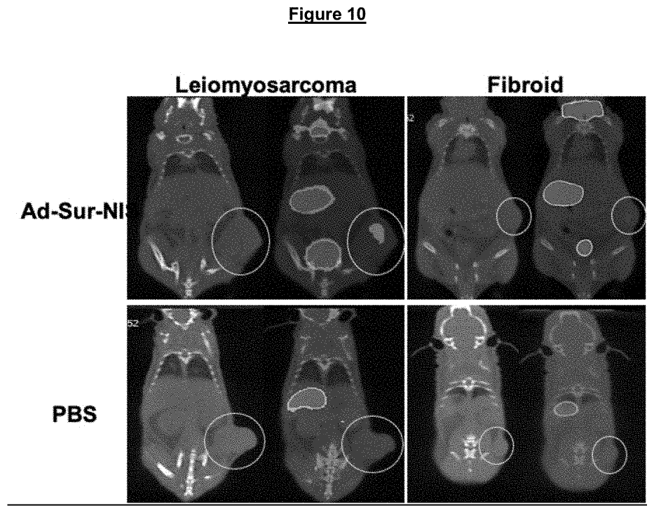

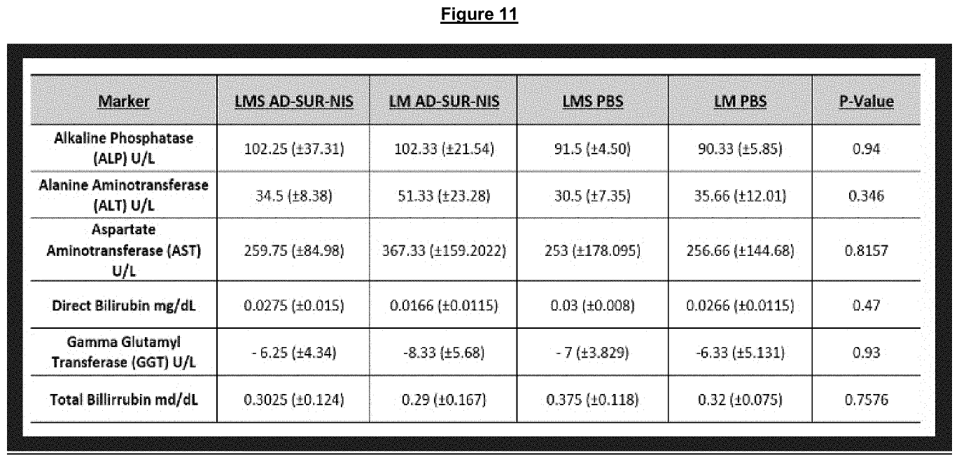

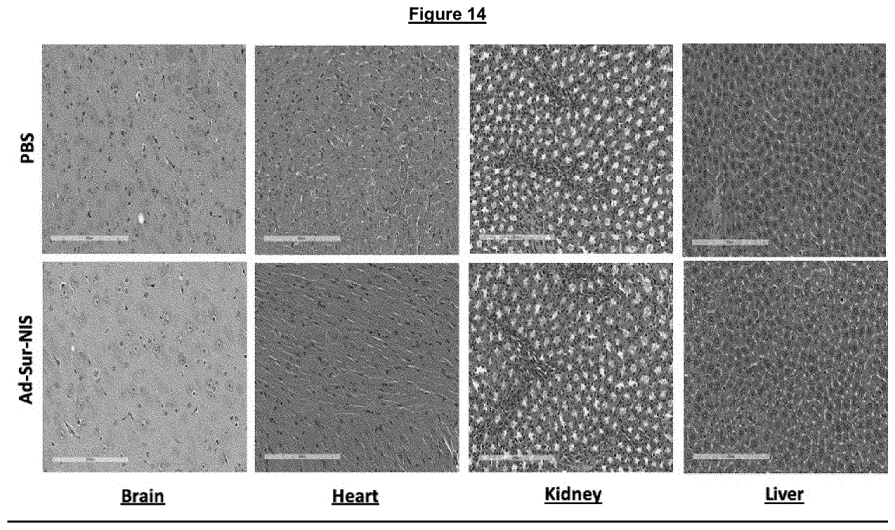

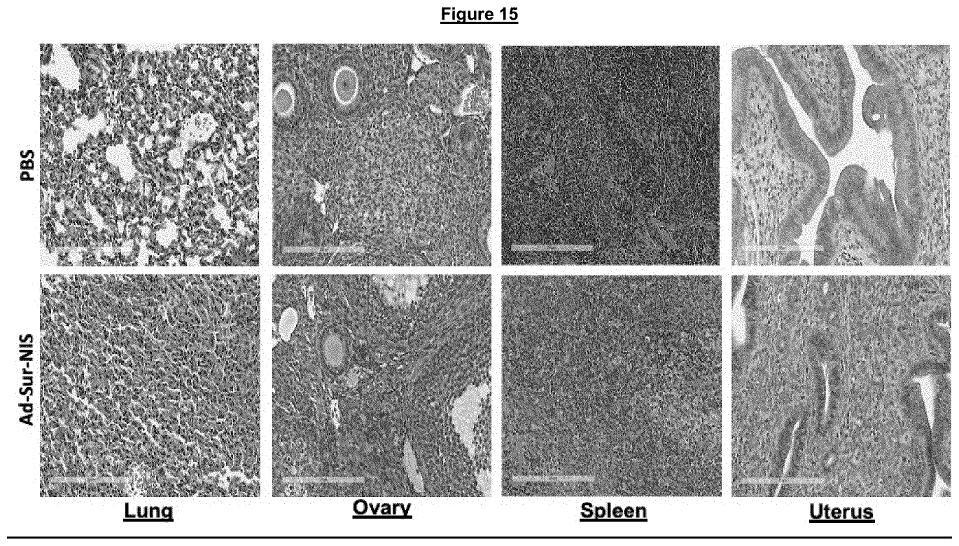

PET/CT scan images from animals LMS AD-Sur-NIS, LM AD-Sur-NIS, LMS PBS, and LM PBS demonstrate the higher expression of NIS in LMS AD-Sur-NIS compare to LM AD-Sur-NIS after F18 administration FIG. 10; circles are tumors). Changes in liver function are demonstrated as shown in FIG. 11 with histopathology in PBS and AD-Sur-NIS mice in the brain, heart, kidney, and liver (FIG. 14) and in the lung, ovary, spleen, and uterus FIG. 15).

These results demonstrate that the radiotracer in combination with Ad-Sur-NIS is able to distinguish uterine sarcomas from fibroids. This allows for noninvasive detection of uterine tumors. Furthermore, the statistically significant decrease in LMS Ad-Sur-NIS indicates the specificity of the composition and methods disclosed herein.

This application refers to various journal articles, and other publications, which are incorporated herein by reference. If there is a conflict between any of the incorporated references and the instant specification, the specification shall control. In addition, any particular embodiment of the present invention set forth herein that falls within the prior art can be explicitly excluded from any one or more of the claims. Because such embodiments are deemed to be known to one of ordinary skill in the art, they can be excluded even if the exclusion is not set forth explicitly herein. Any particular embodiment of the invention set forth herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention set forth herein, as defined in the following claims.

Although specific examples and details are set forth herein, systems and methods consistent with the invention set forth herein of the present disclosure are contemplated within the scope of the disclosure and claims.

1. An adenovirus Survivin-Sodium iodide symporter (Ad-SUR-NIS) vector composition comprising:
a Survivin promoter operatively linked to a sodium iodine symportor protein (NIS) protein-encoding sequence that is capable of infecting leiomyosarcoma (LMS) cells in vivo.

2. The composition of claim 1, wherein in the presence of radiotracer distinguishable imaging data is generated in LMS cells having sufficient energy emitted from the radiotracer that can be captured as imaging data from the LMS, and wherein only minimal energy is emitted from the radiotracer and captured as the imaging data from uterine fibroids (LM).

3. The composition of claim 2, wherein the radiotracer is I¹²⁴.

4. The composition of claim 1, wherein the composition is configured to be administered according to a percentage Ad-SUR-NIS weight dose composition that induces NIS expression allowing for differential detection of LMS.

5-7. (canceled)

8. A method of detecting uterine sarcoma in a woman, the method comprising:
administering to the subject a composition comprising an adenovirus Survivin-Sodium iodide symporter Ad-SUR-NIS vector and a radiotracer;
exposing a uterus of the woman where the presence of a sarcoma is suspected to PET-CT imaging; and
capturing energy emitted from the target area of the subject in the imaging data, wherein the presence of the energy emitted correlates to symporter expression uptake of a radiotracer indirectly indicating the presence of a sarcoma in the target area of the subject.

9. The method of claim 8, wherein the sarcoma is exposed to a weight percent of the composition selected from a uterine mass weight dose range and a radiotracer.

10. The method of claim 8, wherein the composition is administered 0 hours to about 24 hours or more before radiotracer administration.

11. (canceled)

12. A pre-surgical diagnostic system for detecting a leiomyosarcoma (LMS) based on survivin expression in uterine sarcoma of a subject comprising:
an adenovirus Survivin-Sodium iodide symporter (Ad-SUR-NIS) composition configured for delivery to the LMS in a weight dosage concentration selected from a uterine mass weight dose range; and
a radiotracer configured to emit energy based on gene expression of the Ad-SUR-NIS symporter and uptake of the radiotracer thereby; wherein
an imaging device configured to capture energy signals emitted from the radiotracer as an indirect confirmation of the presence of a leiomyosarcoma (LMS).

13. The system of claim 12, wherein the weight dosage indicates the presence of LMS based on reporter gene expression of the radiotracer in PET-CT imaging data.

14-21. (canceled)

22. The method of claim 8 wherein the composition is administered intravenously.

23. The composition of claim 1, further characterized by a uterine mass weight dose range of a viral vector containing a survivin promotor configured to express NIS weight dose composition.

24. A screening kit for noninvasively detecting a cancerous mass comprising:
the composition of claim 1 stored in a suitable delivery vehicle, and optionally,
a radiotracer configured for delivery after the composition, and data access information configured to inform a next procedure based on the energy emitted and displayed in the imaging data.

25. (canceled)

26. A method of detecting sarcoma in a subject, the method comprising the steps of:
administering a composition comprising a viral vector containing a survivin promotor configured to express a sodium iodine symportor protein (NIS) protein in the subject;
delaying administration of a radiotracer for a delay time until the viral vector reaches a target area in the subject to express the NIS protein in the mass;
administering the radiotracer after the delay time, where the radiotracer can be selected from Table 2;
exposing the target area to PET-CT imaging to generate imaging data; and
capturing energy emitted from the target area of the subject in the imaging data, wherein the presence of the energy emitted correlates to uptake of a radiotracer due to NIS expression indicating the presence of a cancerous mass in the target area of the subject.

27. The method of claim 26, wherein exposing the target area comprises exposing the cancerous mass to a selected weight percent of the composition selected from a uterine mass weight dose range.

28. The method of claim 26, wherein the composition is administered from about 8 hours to about 10 days before radiation exposure.

29. The method of claim 26, wherein the energy emitted in imaging data is captured using PET-CT imaging.

30-36. (canceled)
